(19) United States
(12) Patent Application Publication
(10) Pub. No.: US 2009/0068281 A1
(43) Pub. Date: Mar. 12, 2009

(54) Composition, Functional Food and Pharmaceutical Composition for Improvement in Obesity

(76) Inventors: Koji Toyomura, Tsukuba-shi (JP); Mikako Sato, Tsukuba-shi (JP); Jun Ichikawa, Tsukuba-shi (JP)

Correspondence Address: BIRCH STEWART KOLASCH & BIRCH, PO BOX 747, FALLS CHURCH, VA, 22040-0747, US

(21) Appl. No.: 11/887,239

(22) PCT Filed: Mar. 29, 2005
(86) PCT No.: PCT/JP2005/005975

§ 371 Date: Sep. 27, 2007

(51) Int. Cl. A61K 35/60; A23L 1/313; A23L 1/302

(52) U.S. Cl. 424/520; 426/655; 426/72

(57) ABSTRACT

A composition for improving obesity is disclosed. The composition contains a natural ingredient free of side effects in daily intake as an active ingredient. The composition is capable of remarkably reducing body fat, as compared with a conventional ingredient which is derived from a natural ingredient as an active ingredient, and is capable of suppressing fat storage. The composition includes a meat extract or a fish meat extract, and a fat storage suppressive substance.

### TECHNICAL FIELD

The present invention relates to an obesity improving composition containing at least one kind of fat storage suppressive substance in a meat extract and/or a fish meat extract, as well as a functional food and a pharmaceutical composition using the same.

### BACKGROUND ART

Obesity has become a social issue in the modern age. It is conceived that obesity is a result of digestion of high-calorific food, underexercising resulting from development of transportation facility, overeating under stressful conditions, or other factors.

It is preferred to avoid obesity by habitual exercise. Some people, however, need extraordinary efforts in making exercise a habit. Accordingly, success in avoiding obesity through exercise greatly relies on lifestyle habits of individuals.

There have been developed anti-obesity drugs (e.g. Mazindol) in order to avoid obesity from a medical viewpoint.

The anti-obesity drug is adapted to control the food intake capacity by affecting the appetite center in the brain of subjects and suppressing their appetite. The anti-obesity drug has a high efficacy in avoiding obesity.

The anti-obesity drug, is, however, known to cause adverse side effects such as dry mouth, nausea, constipation, sleeplessness, or anxiety. It is also pointed out that administration of the drug to patients with a cardiac disease may worsen their arrhythmic or anginal symptoms.

In view of the above, it is not desirable for people to make a habit of taking the anti-obesity drug for a long time.

A search has been made for an ingredient capable of suppressing storage of fat contained in food or the like, in place of the anti-obesity drug with a possibility of side effects as mentioned above.

Carnosine and anserine included in extracts from poultry, fish meat, or the like are some of the ingredients that have been focused in recent years.

For instance, a patent document D1 discloses a health food supplement, for improving lipid metabolism, which contains an extract from poultry as an active ingredient.

Another patent document D2 discloses a food supplement for improving obesity and for dietary purpose. The food supplement is obtained by electrodialyzing a meat extract composed of an effluent obtained by processing meat and its concentrate in an acidic region or in a neutral region, and concentrating L-carnitine, histidine-related dipeptide (carnosine, anserine, balenine), and taurine, which abundantly exist in meat, as primary ingredients.

Since the extract from poultry or the like is a natural ingredient, the extract is free of side effects, and provides enhanced safety. The extract, however, has a poor effect in avoiding obesity, as compared with the anti-obesity drug. Accordingly, exercise, restriction on daily food intake, or the like is additionally required to drastically reduce body fat.

D1: Japanese Unexamined Patent Publication No. 2000-198739

D2: Japanese Unexamined Patent Publication No. 2000-57869

### DISCLOSURE OF THE INVENTION

It is an object of the invention to provide an obesity improving composition containing, as an active ingredient, an extract from meat or fish meat, which is a natural ingredient free of side effects in daily intake, wherein the composition is capable of remarkably reducing body fat, as compared with a conventional ingredient which is derived from a natural ingredient, and is capable of suppressing fat storage.

The inventors, as a result of extensive examination to solve the above problem, found that a fat storage suppressive effect can be obtained in a synergic manner in the case where an extract from meat or fish meat (hereinafter, called as “extract from meat or the like”) containing carnosine and anserine is taken for a certain period in combination with a specific fat storage suppressive substance, as compared with a condition that solely the extract from meat or the like, or solely the fat storage suppressive substance is taken, and came up with the invention.

### BEST MODE FOR CARRYING OUT THE INVENTION

The following is a description on an obesity improving composition containing a meat extract and/or a fish meat extract, and a fat storage suppressive substance according to the invention.

The meat extract and/or the fish meat extract is obtained by isolating and purifying an extract obtained by a meat or fish meat extracting process, or an effluent to be discharged in meat or fish meat processing.

The meat extract and/or the fish meat extract contains carnosine and anserine having an effect of suppressing storage of body fat.

Among the meat extract and/or the fish meat extract, an extract derived from poultry is particularly preferable, because the poultry extract has a balanced composition ratio in carnosine/anserine as active ingredients, and provides a superior effect in suppressing storage of body fat.

The meat extract and/or the fish meat extract normally contains 3 to 20% by mass of carnosine, and 5 to 30% by mass of anserine, and further contains peptide and amino acid such as taurine, 3-methylhistidine, creatine, or creatinine; and various vitamins.

In the following, a method for producing a poultry extract is described as an example of a method for producing an extract from meat or the like. A meat extract other than the poultry extract, and a fish meat extract can be produced by a similar method as the method for producing the poultry extract.

First, poultry such as chicken meat is chopped, followed by addition of warm water and an acid component or an alkali component. Then the ingredients are subjected to hydrothermal extraction. Thus, an extract is obtained.

The kind of the acid component is not specifically limited, as far as the acid component is an acidic substance that is permitted to be added as a food additive. Examples of the acid component are e.g. hydrochloric acid, citric acid, acetic acid, and ascorbic acid. Among these, citric acid is preferably used.

The kind of the alkali component is not specifically limited, as far as the alkali component is an alkaline substance that is permitted to be added as a food additive. Examples of the alkali component are sodium hydroxide, potassium hydroxide, and triethanolamine. Among these, sodium hydroxide is preferably used.

Preferably, a ratio of the acid component or the alkali component to the poultry mass ranges from 0.01 to 4.0% by mass, and more preferably, from about 0.1 to 2.0% by mass.

The extracting condition is not specifically limited, but the extract is normally processed at a temperature of 95 to 100° C. for about three to six hours.

After the extract is obtained, solid content is removed by subjecting the extract to filtration. Thereafter, an oil component containing cholesterol is removed to purify the filtrate by an ordinary process such as a liquid separation process or a centrifugal process. Then, the extract free of the oil component is demineralized by a well-known demineralization process such as column chromatography or electrodialysis, whereby a purified extract containing the poultry extract is yielded.

Then, the purified extract is concentrated by a well-known vacuum concentration, followed by drying with use of heated air drying, spray drying, freeze drying, or the like. Thus, a poultry extract in the form of powder is obtained.

A process of treating the poultry extract with protease may be additionally provided in an appropriate stage of the aforementioned processes e.g. immediately after the extraction, after the filtration, or after the purification.

The kind of the protease to be used in the protease treatment may be properly selected, and is not specifically limited. Examples of the protease are papain, trypsin, and pepsine. Among these, papain is preferably used.

The protease may be used alone or in combination of two or more.

The inventive composition for improving obesity has a feature that a fat storage suppressive substance is added to the extract from meat or the like.

The fat storage suppressive substance to be used in the invention is a group of substances for suppressing fat storage in the body. Specifically, the fat storage suppressive substance includes: a lipid absorption inhibitor having a property that the substance directly adsorbs lipid and is discharged out of the body without absorbing lipid; a fat decomposition accelerator having a property that the substance is acted on an endocrine system of accelerating lipid decomposition and accelerates decomposition of fat stored in body as body fat; a fat storage inhibitor having a property that the substance reduces neutral fat in blood and inhibits fat storage; a lipid metabolism accelerator having a property that the substance activates an enzyme system of decomposing lipid such as lipase and accelerates decomposition of fat stored as body fat; a lipid synthesis inhibitor acting as a substance for inhibiting the function of a lipid synthesizing system; and a carbohydrate metabolism accelerator having a property that the substance accelerates insulin secretion and suppresses conversion from carbohydrate to lipid.

Examples of the lipid absorption inhibitor are conjugate linoleic acid (CLA); chitin; chitosan; and dietary fibers such as indigestible dextrin, mannan, pectine, or alginic acid. Examples of the fat decomposition accelerator are capsaicin and chlorogenic acid. Examples of the fat storage inhibitor are eicosapentaenoic acid (EPA) and docosahexaenoic acid (DHA). Examples of the lipid metabolism accelerator are catechin; amino acid; vitamins such as vitamin B1, vitamin B2, vitamin B6, or pantothenic acid; carnitine; ubiquinone (CoQ10); and a lipoic acid. Examples of the lipid synthesis inhibitor are hydroxycitric acid (HCA) and garcinia extracts containing HCA. Examples of the carbohydrate metabolism accelerator are chromium-containing substances such as chromic acid or chromium picolinate, and derivatives thereof.

Examples of conjugate linoleic acid as the lipid absorption inhibitor are cis-9,trans-11-octadecadienoic acid, cis-9,cis-11-octadecadienoic acid, trans-9,cis-11-octadecadienoic acid, trans-9,trans-11-octadecadienoic acid, cis-10,cis-12-octadecadienoic acid, cis-10,trans-12-octadecadienoic acid, trans-10,cis-12-octadecadienoic acid, and trans-10,trans-12-octadecadienoic acid.

Examples of catechin as the lipid metabolism accelerator are catechin, gallocatechin, catechin gallate, gallocatechin gallate, epicatechin, epigallocatechin, epicatechin gallate, and epigallocatechin gallate.

Examples of amino acid as the lipid metabolism accelerator are L-amino acid such as lysine, alanine, arginine, or proline; and branched-chain amino acid (BCAA) such as isoleucine, leucine, or valine.

Among the above, conjugate linoleic acid, capsaicin, carnitine, and garcinia extracts containing HCA are preferred in the point of providing a large effect of improving obesity.

The inventive composition for improving obesity may contain, as other ingredients, various active ingredients such as proteins or peptides e.g. hydrolyses of creatine or gelatin, nucleic acids, or glycolipids; a stabilizer such as various antioxidants; an anticoagulant; or generally used additives, according to needs, in addition to the extract from meat or the like, and the fat storage suppressive substance.

A method for producing the inventive composition for improving obesity is not specifically limited. The inventive composition for improving obesity may be produced as a powder composition obtained by uniformly admixing powders of the extract from meat or the like and powders of the fat storage suppressive substance; a granulated composition obtained by granulating the powder composition; a tabletized composition; or a liquid composition obtained by dissolving the fat storage suppressive substance in an extract obtained from poultry or the like or its concentrate, before a drying step is conducted.

The inventive composition for improving obesity obtained in the aforementioned manner may be used as a functional food for improving obesity such as tablet supplements, liquid supplements, beverages, or processed foods; or a pharmaceutical composition for improving obesity by adding other ingredient(s) according to need.

It is expected that obesity can be improved by periodically taking the supplement/food containing the inventive composition for improving obesity, the pharmaceutical composition containing the inventive composition for improving obesity, or an equivalent edible composition.

As the daily intake requirement of the inventive functional food for improving obesity or the inventive pharmaceutical composition for improving obesity, it is preferred to take carnosine in the range from about 0.005 to 5 g, more preferably in the range from about 0.05 to 1 g, and anserine in the range from about 0.005 to 10 g, more preferably in the range from about 0.2 to 2 g in the aspect of suppressing fat storage.

The intake requirement of the fat storage suppressive substance differs depending on the kind of a substance to be used. Specifically, as the daily intake requirement, it is preferred to take capsaicin in the range from 0.01 to 1 mg, vitamins in the range from 0.1 to 20 mg, ubiquinone in the range from 10 to 200 mg, HCA in the range from 100 to 30,000 mg, more preferably 200 to 2,000 mg, chromic acid in the range from 0.01 to 1 mg, and other ingredient(s) in the range from 100 to 30,000 mg.

In the case where the intake requirement of the fat storage suppressive substance is smaller than the lower limit, the fat storage suppressive effect resulting from the extract from meat or the like may less likely to exhibit in a synergic manner. On the other hand, even if the intake requirement exceeds the upper limit, the synergy effect may less likely to be increased, or the effect may be saturated by way of discharge out of the body, or a like phenomenon.

In view of the above, it is preferred to prepare the inventive functional food for improving obesity or the inventive pharmaceutical composition for improving obesity in such an optimal manner that a subject can take the active ingredients with the aforementioned intake requirement.

It is preferred to prepare the inventive composition for improving obesity with such a component concentration/composition ratio that the intake requirement can be easily controlled in producing the functional food for improving obesity or the pharmaceutical composition for improving obesity.

The inventive composition for improving obesity is obtained by adding the fat storage suppressive substance derived from a natural ingredient to the extract from meat or the like containing carnosine and anserine. Accordingly, the inventive composition is free from side effects, unlike currently available chemically-synthesized obesity treatment drugs. Also, as compared with an obesity improving effect expected to be obtained in the case where solely the extract from meat or the like containing carnosine and anserine, or solely the fat storage suppressive substance derived from a natural ingredient is taken, the inventive composition is advantageous in securing the synergy effect of improving obesity by taking the two components in combination.

### EXAMPLES

In the following, the invention is more specifically described by way of examples, but the invention is not limited thereto.

### (Production of Poultry Extract)

A poultry extract was produced by the following process.

Chicken meat was ground by a meat grinder. After warm water of a mass 1.5 times as large as the mass of the chicken meat was added, citric acid of 0.2% by mass was added relative to the mass of the chicken meat so that the specimen attained pH of 5.5.

Then, after the specimen was extracted at a temperature of 90° C. for four hours, insoluble matter of a size not smaller than 100 meshes was removed by filtration, and an extract was obtained.

Then, the extract was freeze-dried, followed by pulverization. Thereby, powders of poultry extract of a size not larger than 50 meshes were obtained.

As a result of analyzing the poultry extract powders by liquid chromatography, the poultry extract powders contained 5% by mass of carnosine, 10% by mass of anserine, and 0.1% by mass of taurine.

The following ingredients were used as the suppressive substance in Examples.

Conjugate linoleic acid or chitosan was used as the lipid absorption inhibitor. Capsaicin or chlorogenic acid was used as the fat decomposition accelerator. EPA or DHA was used as the fat storage inhibitor. Catechin, a mixture containing arginine, ornithine, and lysine with a composition ratio of 1:1:2 (hereinafter, called as “amino acid mixture”), a mixture containing isoleucine, leucine, and valine with a composition ratio of 1:1:2 (hereinafter, called as “branched-chain amino acid”), vitamin B1, vitamin B2, vitamin B6, pantothenic acid, carnitine, or ubiquinone (CoQ10) was used as the lipid metabolism accelerator. Garcinia extract was used as the lipid synthesis inhibitor. Chromic acid was used as the carbohydrate metabolism accelerator.

### Example 1

A beverage was obtained, as a beverage containing the poultry extract and the fat storage suppressive substance, by dissolving the ingredients recited in Table 1 in 150 mL water, while stirring.

TABLE 1

| beverage composition | content (mg) |
|---|---|
| poultry extract powder | 6,000 |
| conjugate linoleic acid | 600 |
| 75%-reduced maltose | 18,000 |
| lemon juice | 150 |
| glycine | 300 |
| citric acid | 225 |
| ascorbic acid | 150 |
| gardenia pigment | 60 |
| flavoring ingredient | 345 |

The beverage was taken one time per day by ten subjects consisting of adult men and women in age from 20's to 50's, whose BMI (Body Mass Index) was 26.4 or more. A reduction rate of body fat of the subjects 90 days after start of taking the beverage was measured by a body fat scale. An average of body fat reduction rate of the ten subjects was calculated by the following equation.

body fat reduction rate (%)=(body fat rate before drinking−body fat rate 90 days after drinking)/(body fat rate before drinking)×100

In assessing the body fat reduction rate with respect to all the Examples including Examples 1 through 17 and Comparative Examples 1 through 19, single blind test was conducted, in which the kind of beverage was not informed to the subjects.

An assessment result in Example 1 is shown in Table 2.

### Examples 2 through 17

Beverages were prepared in a similar manner as Example 1 except that a corresponding substance of the kind and the content recited in Table 2 was included, in place of conjugate linoleic acid of 600 mg. Body fat reduction rates in the respective Examples 2 through 17 were assessed.

An assessment result is shown in Table 2.

### Comparative Examples 1 through 17

Beverages were prepared in a similar manner as Examples 1 through 17 except that the poultry extract was not included. Body fat reduction rates in the respective Comparative through 17 were assessed.

An assessment result is shown in Table 2.

TABLE 2

| | | | POULTRY EXTRACT | | POULTRY EXTRACT | |
|---|---|---|---|---|---|---|
| | | | INCLUDED | | NOT INCLUDED | |
| FAT STORAGE SUPPRESSIVE SUBSTANCE | | | | BODY FAT | | BODY FAT |
| | | REQUIREMENT | EXAMPLE | REDUCTION | COMPARATIVE | REDUCTION |
| GROUP | KIND | (mg/DAY) | NO. | RATE (%) | EXAMPLE NO. | RATE (%) |
| LIPID ABSORPTION | CONJUGATE | 600 | 1 | 34 | 1 | 6 |
| INHIBITOR | LINOLEIC ACID | | | | | |
| | CHITOSAN | 600 | 2 | 20 | 2 | 4 |
| FAT DECOMPOSITION | CAPSAICIN | 0.1 | 3 | 37 | 3 | 7 |
| ACCELERATOR | CHLOROGENIC ACID | 600 | 4 | 31 | 4 | 5 |
| FAT STORAGE | EPA | 600 | 5 | 27 | 5 | 4 |
| INHIBITOR | DHA | 600 | 6 | 28 | 6 | 4 |
| LIPID METABOLISM | CATECHIN | 600 | 7 | 29 | 7 | 7 |
| ACCELERATOR | AMINO ACID MIXTURE | 600 | 8 | 28 | 8 | 6 |
| | BRANCHED-CHAIN | 600 | 9 | 29 | 9 | 5 |
| | AMINO ACID | | | | | |
| | VITAMIN B1 | 1 | 10 | 27 | 10 | 6 |
| | VITAMIN B2 | 1 | 11 | 28 | 11 | 6 |
| | VITAMIN B6 | 1 | 12 | 28 | 12 | 7 |
| | PANTOTHENIC ACID | 1 | 13 | 26 | 13 | 8 |
| | CARNITINE | 600 | 14 | 34 | 14 | 7 |
| | UBIQUINONE | 100 | 15 | 29 | 15 | 6 |
| LIPID SYNTHESIS | HCA | 600 | 16 | 35 | 16 | 5 |
| INHIBITOR | | | | | | |
| CARBOHYDRATE | CHROMIC ACID | 0.1 | 17 | 31 | 17 | 5 |
| METABOLISM ACCELERATOR | | | | | | |

### Comparative Example 18

A beverage was prepared in a similar manner as Example 1 except that the poultry extract powder and conjugate linoleic acid were not included. The body fat rate in Comparative Example 18 was assessed. An assessment result is shown in Table 3.

### Comparative Example 19

A beverage was prepared in a similar manner as Example 1 except that conjugate linoleic acid was not included. The body fat rate in Comparative Example 19 was assessed. An assessment result is shown in Table 3.

TABLE 3

| | | fat storage | body fat |
|---|---|---|---|
| Comparative | poultry | suppressive | reduction rate |
| Example No. | extract | substance | (%) |
| 18 | not included | not included | 1 |
| 19 | included | not included | 6 |

As is obvious from the result shown in Table 3, the subject group who drank the beverage excluding the active ingredients showed a body fat reduction rate of 1% 90 days after start of drinking the beverage (see Comparative Example 18), and the subject group who drank the beverage merely containing the poultry extract showed a body fat reduction rate of 6% 90 days after start of drinking the beverage (see Comparative Example 19).

On the other hand, as is obvious from the result shown in Table 2, whereas the subject group who drank the beverages merely containing the corresponding lipid suppressive substance, as shown in Comparative Examples 1 through 17, showed a body fat reduction rate of at most 4 to 8%, the subject group who drank the beverages containing the poultry extract and the corresponding lipid suppressive substance, as shown in Examples 1 through 17, showed a body fat reduction rate of 20 to 37%.

Thus, the body fat was reduced in a synergic manner by adding the various lipid suppressive substances to the poultry extract.

Observing the body fat reduction effect in use of individual groups of the fat storage suppressive substance i.e. the carbohydrate metabolism accelerator, the lipid absorption inhibitor, the lipid metabolism accelerator, the fat storage inhibitor, and the lipid metabolism accelerator, use of the individual groups shows a superior effect of reducing body fat. Specifically, use of the carbohydrate metabolism accelerator shows a body fat reduction rate of 31%, use of the fat storage inhibitor shows a body fat reduction rate of 27 to 28%, use of the lipid metabolism accelerator shows a body fat reduction rate of 31% and 37%, use of the lipid metabolism accelerator shows a body fat reduction rate of 26 to 34%, and use of the lipid absorption inhibitor shows a body fat reduction rate of 20 to 35%.

1. A composition for improving obesity, comprising a meat extract and/or a fish meat extract, and a fat storage suppressive substance.

2. The composition for improving obesity according to claim 1, wherein the meat extract is a poultry extract.

3. The composition for improving obesity according to claim 1, wherein
the fat storage suppressive substance is at least one kind selected from the group consisting of a lipid absorption inhibitor, a fat decomposition accelerator, a fat storage inhibitor, a lipid metabolism accelerator, a lipid synthesis inhibitor, and a carbohydrate metabolism accelerator.

4. The composition for improving obesity according to claim 3, wherein
the lipid absorption inhibitor is at least one kind selected from the group consisting of conjugate linoleic acid (CLA), chitin, chitosan, indigestible dextrin, mannan, pectine, and alginic acid.

5. The composition for improving obesity according to claim 3, wherein
the fat decomposition accelerator is selected from capsaicin and/or chlorogenic acid.

6. The composition for improving obesity according to claim 3, wherein
the fat storage inhibitor is eicosapentaenoic acid (EPA) and/or docosahexaenoic acid (DHA).

7. The composition for improving obesity according to claim 3, wherein
the lipid metabolism accelerator is at least one kind selected from the group consisting of catechin, amino acid, vitamin B1, vitamin B2, vitamin B6, pantothenic acid, carnitine, ubiquinone (CoQ10), and a lipoic acid.

8. The composition for improving obesity according to claim 3, wherein
the fat synthesis inhibitor is hydroxycitric acid (HCA) and/or a garcinia extract containing hydroxycitric acid (HCA).

9. The composition for improving obesity according to claim 3, wherein
the carbohydrate metabolism accelerator is at least one kind selected from the group consisting of chromic acid, calcium chromate, and chromium picolinate.

10. A functional food for improving obesity containing the composition for improving obesity of claim 1.

11. A pharmaceutical composition for improving obesity containing the composition for improving obesity of claim 1.
